(19) United States
(12) Patent Application Publication
(10) Pub. No.: US 2018/0292300 A1
(43) Pub. Date: Oct. 11, 2018

(54) ROCK STRENGTH EVALUATION DEVICE

(71) Applicant: TOTAL SA, Courbevoie (FR)

(72) Inventors: Kun SU, Idron (FR); Pierre BARLET, Lescar (FR); Edmond POYOL, Idron (FR)

(73) Assignee: TOTAL SA, Courbevoie (FR)

(21) Appl. No.: 15/767,031

(22) PCT Filed: Oct. 9, 2015
(86) PCT No.: PCT/IB2015/002287

§ 371 Date: Apr. 9, 2018

(51) Int. Cl. G01N 3/46; G01N 33/24; G01N 3/58

(52) U.S. Cl. CPC G01N 3/46; G01N 2203/0053; G01N 3/58; G01N 33/24

(57) ABSTRACT

The present invention relates to a rock strength evaluation device including a frame, a cutter support mounted on the frame, the cutter support being rotatable relatively to the frame about a rotation axis; a cutter mounted on the cutter support, and a rock sample support mounted on the frame. At least one of the cutter support and rock sample support is movable relative to one another in a sliding direction and the rotation axis is perpendicular to the sliding direction

### RELATED APPLICATIONS

The present application is a National Phase entry of PCT Application No. PCT/IB2015/002287, filed Oct. 9, 2015, said applications being hereby incorporated by reference wherein in its entirety.

### FIELD OF THE INVENTION

The present invention relates to a rock strength evaluation device, and more particularly to a scratch test device.

### BACKGROUND OF THE INVENTION

The present invention relates to the determination of rock strength parameters, especially to the determination of the rock strength parameters based on scratch tests/with a scratch device.

The approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section. Furthermore, all embodiments are not necessarily intended to solve all or even any of the problems brought forward in this section.

The prior art rock strength evaluation device used for scratch testing and for the determination of rock strength parameters measures only the parameter parallel to the axis of a core.

The prior art scratch device has been developed in the latter 1990's at the University of Minnesota (U.S. Pat. No. 5,670,711 A).

In the scratch device of the prior art, the cutter is linearly translated/moved relative to the rock sample (parallel to the axis of the core of the rock sample) at a constant depth while the forces on the cutter are measured.

The cutter is fixed on a rigid frame in order to be able to accurately measure the various forces to be measured: indeed, the vertical and horizontal forces applied on the cutter for cutting a fixed depth (0.1 mm˜1 mm) on the core's surface lead to determine the rock strength parameters.

Equivalent uniaxial compressive strength (UCS) and internal frication angle can be derived from the vertical force and horizontal force recorded (Mitaim et al. 2004).

These parameters are considered as rock strength parameters parallel to the core since the force applied in front of the cutter is in the direction of the axis of the core, which is the direction of the movement of the cutter.

Nevertheless, these devices and these methods for the prior art only give information on the rock strength parameters in a single direction: the direction of the rock sample (i.e. the direction of the axis of core). Rock strength parameters in other directions are ignored.

It is well known that most of rocks have anisotropy strength parameters, especially for shale and gas shale. For instance, the strength parameters parallel to the bedding plane could be much higher than the strength perpendicular or at 45°.

Strengths parameters in these various directions are important input parameters for study of wellbore stability, gas shale hydraulic fracturing, assessment of risk of sanding production, etc.

Sampling cylindrical samples at different directions from shale cores is a very delicate operation because of high risk of rupture of sample due to weak planes presented in shale. Moreover, the high of cylindrical sample extracted in the direction perpendicular to the axis of core is limited by the diameter of core. In lots of case, the ratio of 2 of high to diameter of cylindrical sample, requested by the protocol of rock mechanics test, can't be reached.

Being able to determine the rock strength parameters in various directions may be advantageous as the rock could have marked anisotropic mechanical properties.

The invention relates to a rock strength evaluation device including:
a frame,
a cutter support mounted on the frame, the cutter support being rotatable relatively to the frame about a rotation axis;
a cutter mounted on the cutter support, and
a rock sample support mounted on the frame.

At least one of the cutter support and rock sample support is movable relative to one another in a sliding direction.

In addition, the rotation axis is perpendicular to the sliding direction.

Therefore, this device/apparatus enables an assessment of the rock strength parameters of the rock samples in at least one direction, this direction is not the standard sliding direction of the rock strength evaluation device of the prior art.

By having a rotating cutter support, it is then possible to have a compact/portable device for assessing the rock strength parameters and without the need to extract cylindrical samples in various directions in the subsoil.

The cutter support may be a disc or a plate or any other support.

In a specific embodiment, at least one of the cutter support and rock sample support being movable in a translating direction relative to the other, the translating direction may be perpendicular to the rotation axis, the translating direction being different from the sliding direction.

This additional translation in a translating direction (which is not the sliding direction) enables increasing the number of directions used for the assessments of the rock strength parameters.

In addition, the cutter may be rotatable relatively to the cutter support, the cutter being adapted to be rotationally moved of a first angle when the cutter support is rotationally moved of a second angle, the first angle being an opposite of said second angle.

This feature enables keeping the cutter in a given direction regardless the rotational position of the cutter support.

Other features and advantages of the apparatus disclosed herein will become apparent from the following description of non-limiting embodiments, with reference to the appended drawings.

### BRIEF DESCRIPTION OF THE DRAWINGS

The present invention is illustrated by way of example, and not by way of limitation, in the figures of the accompanying drawings, in which like reference numerals refer to similar elements and in which:

FIG. 1a and FIG. 1b are side views of two different configurations of a scratch device according to one embodiment of the invention;

FIG. 2a and FIG. 2b are planar elevational views of two different configurations (the same configurations of respectively FIG. 1a and FIG. 1b) of the scratch device according to one embodiment of the invention;

FIG. 3 is a planar elevational view of an example of a circular scratch test performed with the scratch device according to one embodiment of the invention;

FIG. 4 is a planar elevational view of an example of a detail of the scratch device according to one embodiment of the invention in order to maintain the scratch cutter in the same direction.

### DESCRIPTION OF PREFERRED EMBODIMENTS

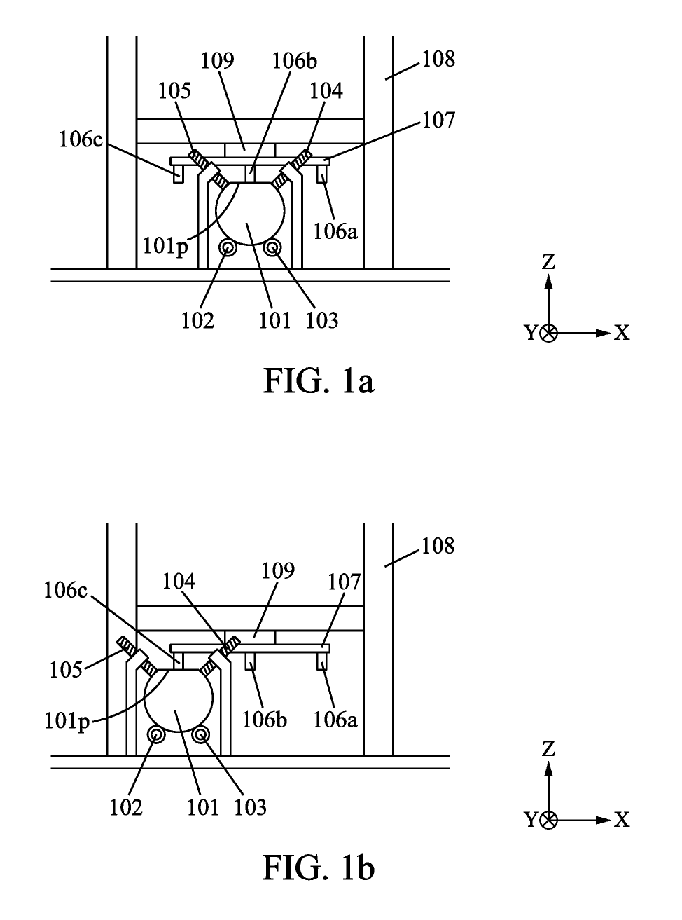

FIG. 1a and FIG. 1b are two different configurations of a scratch device according to one embodiment of the invention (side view).

In these figures, a rock sample 101 lays on two rigid horizontal supports 102 and 103 (namely rock sample support which can have a plurality of forms).

The rock sample 101 is maintained in position thanks to a plurality of screws (only two screws 104 and 105 are shown in these figures due to the perspective).

In addition, a frame 108 may be translated along the core axis of the rock sample (i.e. \vec{y} for said embodiment). Alternatively, the rock sample support may be translated while the frame remains still. A plate (i.e. cutter support)107 is firmly fastened with this frame 108 thanks to a non-deformable piece 109. Nevertheless, the plate 107 may rotate around the vertical \vec{z} axis.

The plate 107 comprises at least one cutter 106a which are not aligned with the rotational axis of the plate 107. For instance, the distance between the cutter and the rotational axis is greater than 3 cm. In addition, the plate 107 may comprise other cutters (106b or 106c) on different points of the plate 107. For instance, a cutter 106b may be aligned with the rotational axis of the plate while another cutter 106c is installed on a point of the plate 107, said point being a symmetric point of the installation point of the cutter 106a about the rotational axis of the plate.

Advantageously, the piece 109 is of the dimension of the plate 107 (e.g. the diameter of piece 109 may be 25% to 100% of the diameter of the plate 107) in order to avoid any deformation of the plate 107 while forces are applied on the cutter 106a, 106b or 106c.

In addition, and referring to FIG. 1b, the frame 108 may be translated along the \vec{x} axis.

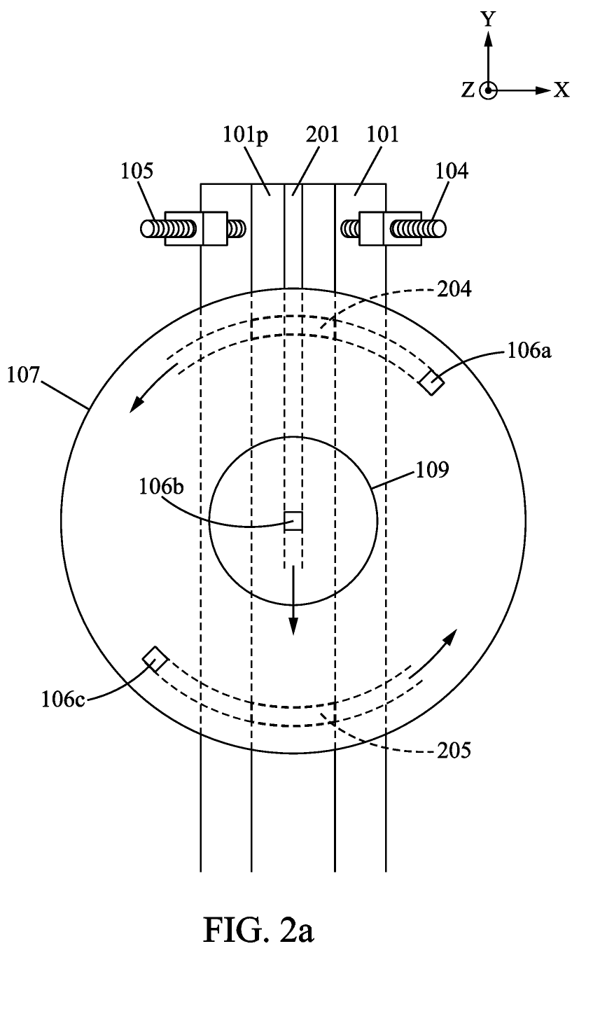

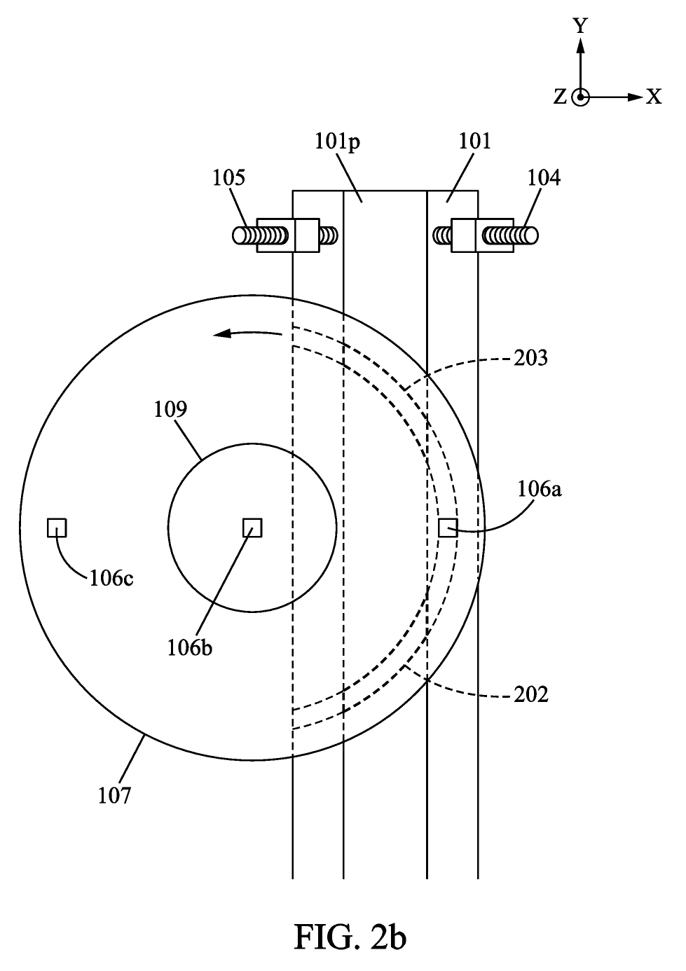

FIG. 2a and FIG. 2b are two different configurations (the same configurations of respectively FIG. 1a and FIG. 1b) of the scratch device according to one embodiment of the invention (plane elevation).

When the frame 108 is centered on the rock sample (i.e. FIG. 2a), the cutter 106b may be in contact of the rock sample 101 (in the plan/prepared zone 101p). Thus, by a translation along the \vec{y} axis (namely the sliding axis), the rock strength parameters may be assessed in that direction (see scratch mark 201).

In addition, by stopping the translation of the frame 108, and by rotating the plate 107, the cutters 106a and 106c may be in contact of the rock sample 101. Thus, by a rotation of the plate 107, the rock strength parameters may be assessed in directions close to the \vec{x} axis (see scratch marks 204 and 205) and not only along the rock sample main direction (\vec{y} axis).

When the frame 108 is not centered on the rock sample (i.e. FIG. 2b), it is possible to rotate the plate 107 and thus the cutters 106a and 106c may be in contact of the rock sample 101 (see scratch marks 202 and 203). The rock strength parameters may thus be assessed in various directions (the directions of the scratch marks). These various directions are functions of the distances of the center of the plate 107 and the axis of the rock sample main direction (\vec{y} axis).

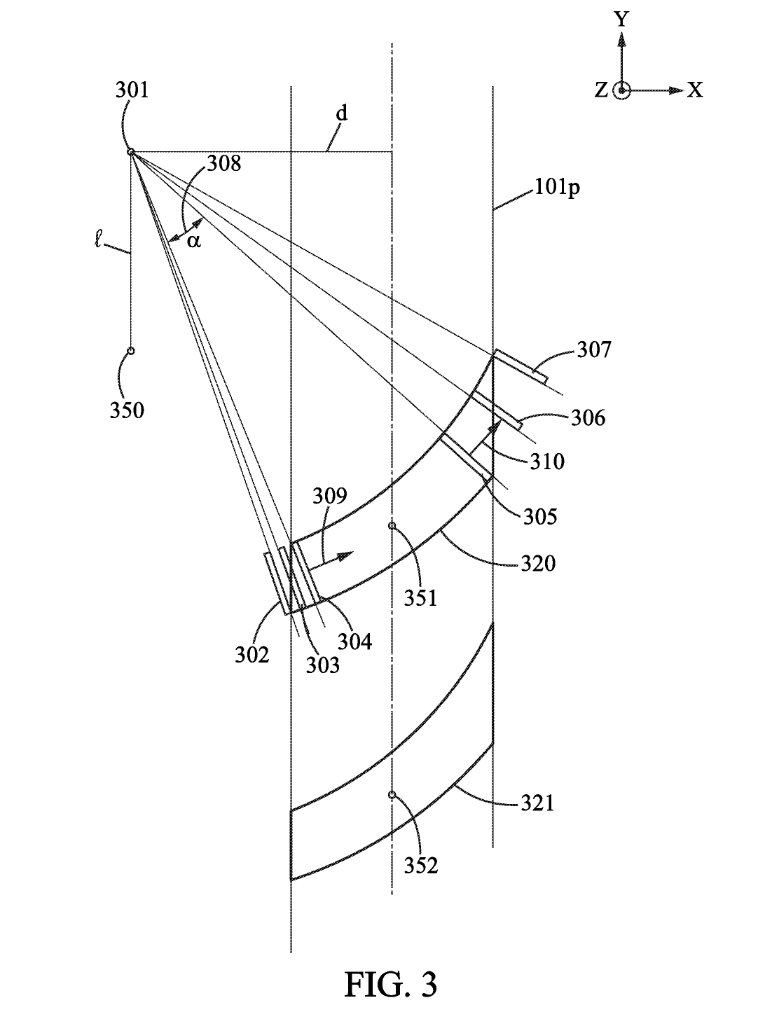

FIG. 3 is an example of a circular scratch test performed with the scratch device according to one embodiment of the invention (plane elevation).

The FIG. 3 may be a zoom on the scratch 202 or 203 of FIG. 2b.

In the present example, the rock sample has two scratches 320 and 321. These scratches are performed with the above mentioned device with the same distance d between the center of the plate 107 and the axis of the rock sample main direction (\vec{y} axis). Thus, it is possible to perform a plurality of rocks strength parameter assessment with the same configuration by translating the cutter support 107 along the \vec{y} axis (e.g. the plate is translated by a vector defined by the points 351 and 352 or by points 301 and 350, distance 1). Therefore, it is possible to compute a mean of all the assessments performed with the same configuration to obtain an accurate estimation of the rock strength parameters in a given direction.

Due to the specific shape of the rock sample (which may have been planned/prepared, see zone 101p), the cutters (either 106a or 106b or 106c) may be not in full contact with the rock sample. For instance:
the cutter in position 302 (or 307) is in contact with the rock sample only by one edge of the cutter ;
the cutter in position 303 (or 306) is in contact with the rock sample only by one half of the cutter.

On the contrary, if the cutter is within a specific angle (308), the cutters may be fully in contact with the rock sample (between the positions 304 and 305).

In one possible embodiment, the rock strength parameters are assessed for each individual scratching direction (e.g. direction 309 for the position 304 of the cutter and direction 310 for the position 305 of the cutter). Thus, mean parameters may be computed for each individual direction of all scratches (320, 321) performed for a same configuration.

In one other possible embodiment, the rock strength parameters are assessed for a mean direction of the scratches (i.e. means of directions 309, 310, etc.). In addition, mean parameters may be computed for all scratches (320, 321) performed for a same configuration.

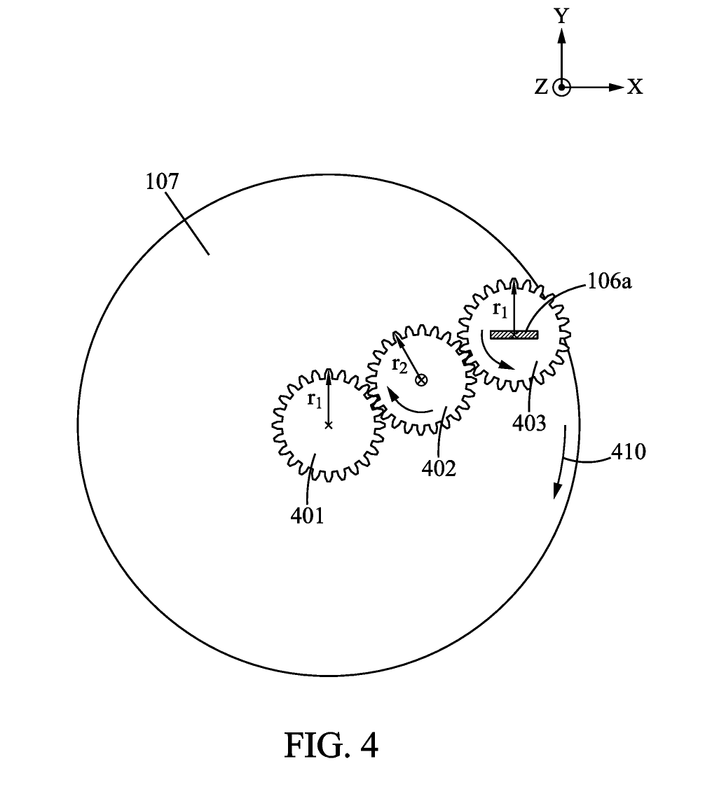

FIG. 4 is an example of a detail of the scratch device according to one embodiment of the invention (plane elevation) in order to maintain the scratch cutter in the same direction.

As it is shown on FIG. 3, the cutters are fixed on the plate: theirs angles with the main axis of the rock sample may thus vary. In addition, during a scratching, the distance followed by the part of the cutter close to the center of the plate 107 is less longer that the distance followed by the part of the cutter close to the edge of the plate 107. Therefore, it may be complex (the rock strength parameters are function of the volume of the rock sample that is removed during the scratches) and inaccurate (the forces applied on the cutters are not uniform) to use the standard equations used for assessing the rock strength parameters during a linear scratch test.

Thus, it may be advantageous to change dynamically the angle of the cutters during the rotation of the plate 107, so that the angle formed between the cutter and the main axis of the rock sample is constant.

To do so, many options are available.

One of the options is to fix gears 401 (radius r₁) in the center of the plate 107: these gears 401 do not move when the plate 107 rotates (direction 410).

Other gears 403 (radius r₁) are attached to the cutter so that the cutter 106a may rotate when the gears 403 rotates. The cutter 106a may be attached to the gears 403 so that the cutter crosses the center of the gears 403.

Gears 402 (radius r₂ which may different from r₁) may connect the gears 401 and the gears 403. Thus, when the plate 107 rotates, the cutter 106a remains parallel.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

A person skilled in the art will readily appreciate that various parameters disclosed in the description may be modified and that various embodiments disclosed may be combined without departing from the scope of the invention.

1. A rock strength evaluation device including:
a frame
a cutter support mounted on the frame, the cutter support being rotatable relatively to the frame about a rotation axis;
a cutter mounted on the cutter support, and
a rock sample support mounted on the frame,
at least one of the cutter support and rock sample support being movable relative to one another in a sliding direction,
wherein the rotation axis is perpendicular to the sliding direction.

2. A device according to claim 1, wherein at least one of the cutter, support and rock sample support being movable in a translating direction relative to the other, the translating direction being perpendicular to the rotation axis, the translating direction being different from the sliding direction.

3. A device according to claim 1, wherein the cutter is rotatable relatively to the cutter support, the cutter rotationally moved to a first angle when the cutter support is rotationally moved to a second angle, the first angle being an opposite of said second angle.
